(19) United States
(12) Patent Application Publication
(10) Pub. No.: US 2005/0020965 A1
(43) Pub. Date: Jan. 27, 2005

(54) Devices and methods for delivering agents to tissue region while preventing leakage

(75) Inventors: Robert F. Rioux, Ashland, MA (US); Steve Anderson, Worcester, MA (US); Paul DiCarlo, Middleboro, MA (US)

Correspondence Address: Bingham McCutchen, LLP, Suite 1800, Three Embarcadero, San Francisco, CA, 94111-4067, US

(73) Assignee: Scimed Life Systems, Inc., Maple Grove, MN (US)

(21) Appl. No.: 10/926,853

(22) Filed: Aug. 25, 2004

Related U.S. Application Data
(63) Continuation-in-part of application No. 10/392,545, filed on Mar. 20, 2003.

(51) Int. Cl. A61N 1/30

(52) U.S. Cl. 604/21; 606/41

(57) ABSTRACT

An apparatus for delivering a therapeutic or diagnostic agent to a target site within a body includes an outer tubular body, an inner tubular body slidably disposed within the outer tubular body, a substance delivery port located on the inner tubular body, and an electrode secured to the outer tubular body. The delivery port can be used to deliver a medical substance to the target site, and the electrode can be used to seal an access channel leading to the treatment site, thereby substantially preventing migration of material from the delivery site. In order to minimize the profile of the apparatus, a braided conductor can extend through the wall of the outer tubular body, thereby providing a means for delivering electrical energy to the electrode, while strengthening the wall of the outer tubular body to minimize the risk of rupture during delivery of the substance.

### RELATED APPLICATION DATA

This application is a continuation-in-part of U.S. patent application Ser. No. 10/392,545, filed on Mar. 20, 2003, the disclosure of which is expressly incorporated by reference herein.

### FIELD OF THE INVENTION

The field of the invention relates to medical devices, and more particularly, to apparatus and methods for delivering therapeutic or diagnostic agents to a site within a body.

### BACKGROUND

Media delivery devices, such as medical needles and catheters, have been used to deliver therapeutic or diagnostic agents to a target site within a body for treatment or diagnostic purposes.

Needles typically have a rigid tubular body for delivering an agent, and a sharp distal tip for puncturing skin and/or other bodily tissues, thereby creating a needle tract through intervening tissues between the skin and the target site. Before the tip of the needle reaches the target site, i.e., while the needle is penetrating or passing through the generally healthy intervening tissue, there is a risk that the agent may leak out from the needle and into the intervening tissue. Since the agent may be sclerotic, necrotic, and/or toxic to living tissue, if the agent leaks or spreads, it may damage the intervening tissue.

After an agent is delivered to the target site, the needle is typically withdrawn, thereby leaving the created tract in the tissues, which eventually closes up through normal healing. However, before the tract is healed, the agent(s) delivered to the target site may leak into the tract, possibly spreading the agent(s) to surrounding tissue. As discussed previously, since the agent may be toxic to living tissue, allowing the agent to spread may damage the surrounding tissue. For example, when treating a prostate with Ethanol, significant amounts of the infused Ethanol may leak through the needle tract, possibly damaging unintended tissue.

Furthermore, when a needle is used to deliver an agent to a tumor, tumor cells may be released into surrounding tissue simply by perforating the tumor with the needle. For example, tumor cells may migrate into the needle tract and into surrounding healthy tissue through the needle tract. This phenomenon is known as “tract seeding.”

Catheters generally have a flexible tubular body, which can be steered or guided to a target site through blood vessels. In a treatment procedure to treat tumor, a catheter can be steered or guided to a tumor site through blood vessels, and be used to deliver a toxic agent to kill tumor cells at the site. However, the same or similar problems described previously with reference to the needles also exist for the catheters. Particularly, before the distal end of the catheter reaches the target site, e.g., while the catheter is passing through the surrounding healthy tissue (e.g., blood vessel), there is a risk that the agent may leak out from the catheter and into the surrounding tissue. As discussed previously, since the agent may be sclerotic, necrotic, and/or toxic to living tissue, if the agent leaks or spreads, it may damage the surrounding healthy tissue. Also, after the toxic agent(s) is delivered to the target site, the toxic agent(s) may leak into the blood vessel that provides access for the catheter, possibly spreading the agent(s) to surrounding tissue, and damaging the surrounding tissue.

In addition, in many applications, it is desirable to use a fluid delivery catheter that has a small cross-sectional dimension, such that the catheter can be inserted through narrow passages, such as blood vessels. However, fluid delivery catheters generally require a certain minimum wall thickness to prevent the catheter from rupturing due to fluid pressure within a delivery lumen of the catheter. As such, it remains a challenge to design a catheter that has a small cross-sectional dimension, but yet, has sufficient wall strength to prevent damage of the wall due to fluid pressure within the lumen.

Thus, apparatus and methods for delivering an agent to a site while preventing or limiting potential leakage of the agent and/or migration of tumor cells to surrounding tissue would be useful.

### SUMMARY OF THE INVENTION

In accordance with a first aspect of the present invention, an apparatus for delivering a medical substance to a target site within a body is provided. The apparatus comprises an outer tubular body and an inner tubular body slidably disposed within the lumen of the outer tubular body, such that the distal end of the inner tubular body can be deployed from the distal end of the outer tubular body. The outer tubular body can either be rigid or flexible, depending on the location of the target site. In one embodiment, the inner tubular body is a needle.

The apparatus further comprises a substance delivery port located on the distal end of the inner tubular body in fluid communication with the second lumen. In this manner, the inner tubular body can be used to deliver a substance to the target site. To this end, a source of medical substance (e.g., a therapeutic or diagnostic agent) can be coupled to the second lumen. The apparatus further comprises a first electrode secured to the distal end of the outer tubular body. In one embodiment, the first electrode is an ablation electrode that can be used, e.g., to seal the channel used to access the target site. The slidable relationship between the inner and outer tubular bodies allows the inner tubular body to be retracted within the outer tubular body, thereby preventing hindrance of the channel ablation. Optionally, a second electrode can be secured to the distal end of the inner tubular body, so that an ablation created by the agent delivery device can be controlled in a more effective manner by moving the inner and outer tubular bodies relative to each other to adjust the distance between the electrodes. The apparatus may optionally comprise an aspiration port, e.g., to remove residual substance from the target site.

In accordance with a second aspect of the present invention, a method of delivering a medical substance (e.g., a therapeutic or diagnostic agent) to a target site (e.g., a tumor) within a body. The method comprises advancing a tubular body through a channel to the target site. The channel can be formed by the tubular body during introduction into the patient (e.g., a needle tract), or can be preexisting (e.g., a lumen of a blood vessel or medullary canal of a bone). The method further comprises delivering the substance via the tubular body to the target site, and delivering ablation energy to seal the channel, whereby material (e.g., a toxic fluid, which may be the delivered substance or tumor cells) is prevented from substantially migrating from the target site. Optionally, the method may further comprise aspirating at least some of the delivered substance from the channel or target site.

In accordance with a third aspect of the present inventions, a medical probe is provided. The probe comprises a probe shaft having a tubular wall and a lumen. The probe shaft may be formed of a single tubular body, or alternatively, multiple tubular bodies, e.g., an outer tubular body formed by the tubular wall, and an inner tubular body that defines the lumen. In one embodiment, the probe shaft is flexible.

The probe further comprises a first electrode secured to the distal end of the probe shaft, and a substance delivery port at the distal end of the probe shaft in fluid communication with the lumen. If the probe shaft has an outer tubular body and an inner tubular body, the electrode can be secured to the outer tubular body, whereas the delivery port can be located on the inner tubular body. Optionally, a second electrode can be provided, in which case, it may be secured to the inner tubular body.

The probe further comprises a braided conductor extending through the wall of the probe shaft in electrical communication with the first electrode. Thus, it can be appreciated that the braided conductor strengthens the shaft wall, while providing a means for delivering electrical energy to the electrode, thereby minimizing the profile of the probe and minimizing the risk of probe rupture during delivery of a substance.

Other aspects and features of the invention will be evident from reading the following detailed description of the preferred embodiments, which are intended to illustrate, but not limit, the invention.

### BRIEF DESCRIPTION OF THE DRAWINGS

The drawings illustrate the design and utility of preferred embodiments of the present invention, in which similar elements are referred to by common reference numerals. In order to better appreciate how advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not intended to limit its scope, the invention will be described and explained with additional specificity and detail through the use of the accompanying drawings.

FIG. 1 is a cross-sectional side view of an agent delivery device constructed in accordance with an embodiment of the invention;

FIG. 2 is a cross-sectional side view of the device of FIG. 1, particularly showing an inner tubular body extending distally relative to an outer tubular body;

FIG. 3 is a cross-sectional side view of a variation of the device of FIG. 1, particularly showing an electrode secured to a different location;

FIG. 4 is a cross-sectional detail of a variation of an inner tubular body used in the device of FIG. 1;

FIGS. 5A-5D are cross-sectional views showing a method for using the apparatus of FIG. 1 to deliver an agent into tissue;

FIG. 6 is a cross-sectional view of another agent delivery device constructed in accordance with an embodiment of the invention;

FIG. 7 is a cross-sectional view of still another agent delivery device constructed in accordance with an embodiment of the invention;

FIG. 8 is a cross-sectional view of yet another agent delivery device constructed in accordance with an embodiment of the invention;

FIG. 9 is a cross-sectional view of yet another agent delivery device constructed in accordance with an embodiment of the invention;

FIG. 10 is a cross-sectional view of yet another agent delivery device constructed in accordance with an embodiment of the invention; and

FIGS. 11A-11C are cross-sectional views showing a method for using the device of FIG. 6 to deliver an agent into tissue.

### DETAILED DESCRIPTION OF THE ILLUSTRATED EMBODIMENTS

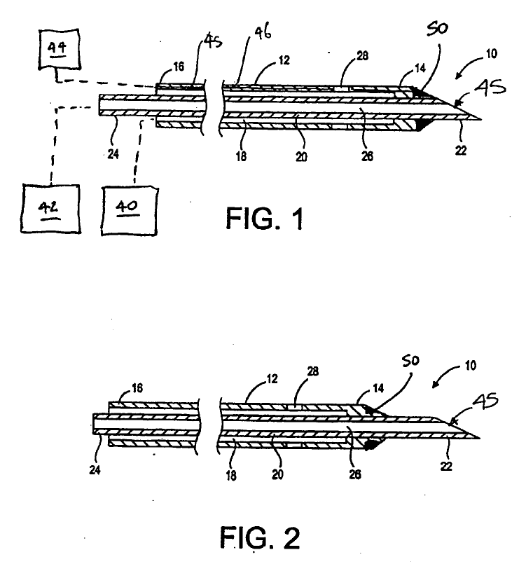

FIG. 1 shows an agent delivery device 10 constructed in accordance with an embodiment of the present invention. The agent delivery device 10 includes an outer tubular body 12 having a proximal end 16, a distal end 14, and a lumen 18 extending between the proximal and distal ends 16, 14. The agent delivery device 10 also includes an inner tubular body 20, such as a rigid needle, positioned coaxially within the lumen 18 of the outer tubular body 12. The inner tubular body 20 has a distal end 22 (which may have a tissue piercing tip and/or a low profile to facilitate penetrating the inner tubular body 20 through skin or other bodily tissues), a proximal end 24, and a lumen 26 extending between the distal and the proximal ends 22 and 24.

The outer and inner tubular bodies 12, 20 may be made from a variety of materials, such as plastics, polymers, metals, alloys, and graphite. It should be understood by those skilled in the art that the flexibility or stiffness of the agent delivery device 10 may be varied by using different materials for the outer and inner tubular bodies 12, 20. The inner tubular body 20 is axially slidable relative to the outer tubular body 12. FIG. 2 shows the inner tubular body 20 advanced distally relative to the outer tubular body 12. The agent delivery device 10 may include a stop (not shown), e.g., secured to the proximal end 24 of the inner tubular body 20 and/or the outer tubular body 12 to prevent the inner tubular body 20 from being advanced beyond a predetermined distance relative to the outer tubular body 12. In the illustrated embodiment, the distal end 14 of the outer tubular body 12 has a cross section that is thicker than the rest of the outer tubular body 12, thereby maintaining the inner tubular body 20 substantially coaxially within the lumen 18 of the outer tubular body 12.

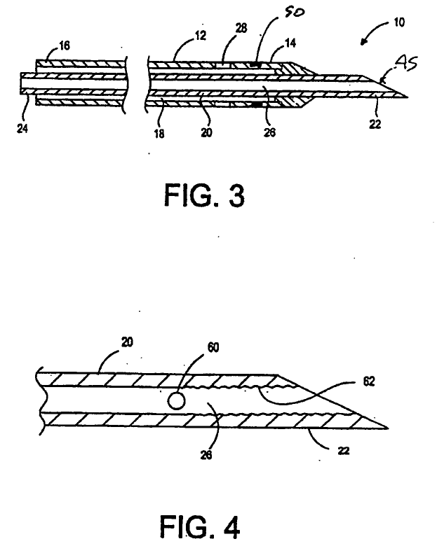

As shown in the illustrated embodiment, the agent delivery device 10 further includes an electrode 50 carried at the distal end 14 of the outer tubular body 12, and a wire 45 disposed within a wall 46 of the outer tubular body 12 in electrical contact with the electrode 50. The electrode 50 may be used to treat tissue in a monopolar or bipolar manner, as is known in the art. A RF connector (not shown), within which the wire 45 proximally terminates, is provided at the proximal end 16 of the outer tubular body 12, so that the electrode 50 can be electrically connected to a radio frequency (RF) generator 44. In the illustrated embodiment, the electrode 50 is located at a distal tip of the outer tubular body 12. However, in alternative embodiments, the electrode 50 can be located at other positions along the outer tubular body 12 (FIG. 3),

The agent delivery device 10 further comprises an optional suction or aspiration port 28 located at or near the distal end 14 of the outer tubular body 12. The aspiration port 28 communicates with the lumen 18 of the outer tubular body 12 (i.e., within the annular space between the outer tubular body 12 and the inner tubular body 20, that is substantially isolated from the lumen 26 of the inner tubular body 20). When a vacuum is created within the lumen 18 of the outer tubular body 12, fluid or objects outside the outer tubular body 12 may be aspirated into the lumen 18 through the aspiration port 28. A vacuum inlet (not shown), with which the lumen 18 communicates, is provided at the proximal end of the outer tubular body 12, so that the aspiration port 28 can be placed into fluid communication with a source of vacuum 40. Any source of vacuum 40, e.g., a syringe, a vacuum line, or a pump, may be used, and is generally well known in the art.

The vacuum inlet may take the form of any suitable device that allows the lumen 18 to be placed into fluid communication with the vacuum 40. For example, the proximal end 16 of the outer tubular body 12 may include a connector, e.g., a male or female luer lock connector (not shown), that may substantially seal the lumen 18 at the proximal end of the outer tubular body 12 when connected to the source of vacuum 40. A section of tubing and the like that communicates with the source of vacuum may include a complementary connector that may engage the connector on the proximal end 16 of the outer tubular member 12. Alternatively, the proximal end 16 of the outer tubular member 12 may be closed, and a nipple or other side port may be provided on the outer tubular member 12 that communicates with the lumen 18. The manner in which the source of vacuum 40 is coupled to the proximal end 16 is not critical to the present invention.

The agent delivery device 10 further comprises an agent delivery port 25 located at or near the distal end 22 of the inner tubular body 20. The delivery port 25 communicates with the lumen 26 of the inner tubular body 20. When a substance is distally conveyed through the lumen 26 of the inner tubular body 20, it exits the delivery port 25. A delivery inlet (not shown), with which the lumen 26 communicates, is provided at the proximal end of the inner tubular body 20, so that the delivery port 25 can be placed into fluid communication with a source 42 of therapeutic or diagnostic agent, which may include a chemical agent, genetic material, or implantable cells as in gene/cell therapy. For example, the proximal end 24 of the inner tubular body 20 may include a connector (not shown) that may be coupled to a syringe, bottle, bag, or other container including the agent therein.

FIG. 4 shows a variation of the inner tubular body 20 that includes one or more delivery ports 60 located along a side wall of the inner tubular body 20. The delivery port(s) 60 is(are) preferably located at or near the distal end 22 of the inner tubular body 20 for delivering an agent therethrough. The delivery port(s) 60 may have different shapes other than the circular shape shown in the illustrated embodiment. For example, the delivery port(s) 60 may have an elliptical shape, rectangular shape, or other customized shape. In addition or alternatively, the interior surface 62 of a distal portion of the lumen 26 of the inner tubular body 20 may be textured (i.e., roughened), which may allow tissue that enters into the distal portion of the lumen 26 to be secured therein and/or retrieved, e.g., while the agent is being delivered through the delivery port(s) 60.

The agent delivery device 10 may include one or more radio-opaque markers (not shown) carried at its distal end, such as at the distal end 22 of the inner tubular body 20 and/or at the distal end 14 of the outer tubular body 12. The radio-opaque marker(s) may assist monitoring the agent delivery device 10 as it is manipulated or positioned during a procedure, as is known in the art.

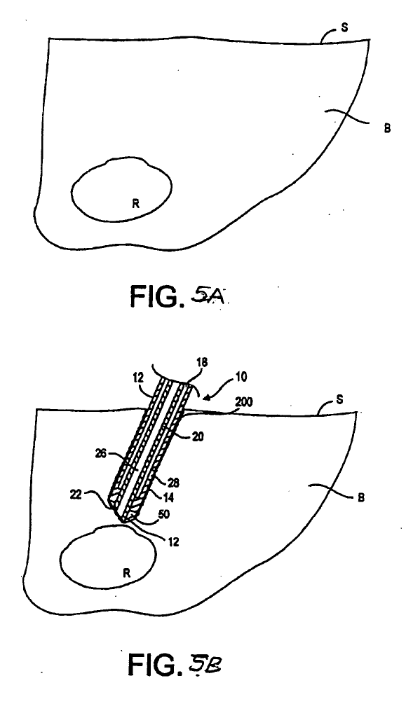

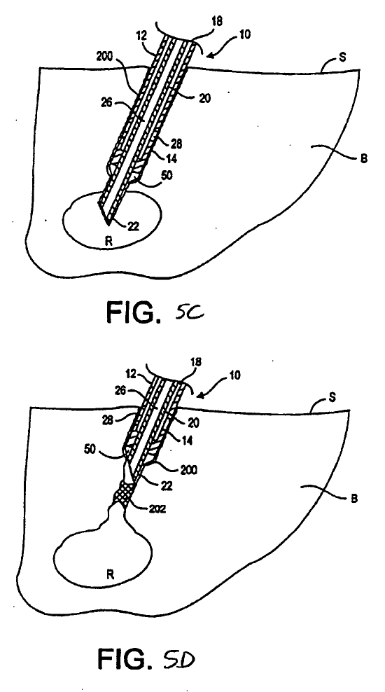

Referring now to FIGS. 5A-5D, the agent delivery device 10 may be used to treat or diagnose a target region R within tissue located beneath the skin S and intervening tissue B of a patient. FIG. 5A shows the region R before the procedure is begun. Before the procedure, the proximal end 24 (not shown) of the inner tubular body 20 may be coupled to a source of agent (also not shown), RF generator (also not shown), and/or a source of vacuum (also not shown).

As shown in FIG. 5B, the distal end 22 of the inner tubular member 20 may be retracted at least partially into the distal end 14 of the outer tubular member 12. The device 10 is then advanced through the skin S and intervening tissue B until the distal ends 22, 14 are located adjacent to the region R. Preferably, the sharp distal end 22 of the inner tubular body 20 facilitates penetrating the skin S and intervening tissue B, thereby creating a tract or pathway 200 leading to the region R.

As shown in FIG. 5C, once the distal end 14 of the outer tubular body 12 is positioned adjacent to the region R, the distal end 22 of the inner tubular body 20 is advanced distally into the region R. Alternatively, the distal end 14 of the outer tubular body 12 may be positioned within the region R, and the inner tubular body 20 may be advanced such that the distal end 22 extends further into the region R. If the agent delivery device 10 includes one or more radio-opaque markers, the marker(s) may be used to assist positioning the distal ends 14, 22 of the agent delivery device 10. Optionally, as shown in FIG. 4, if the inner tubular body 20 includes a textured interior surface 62, tissue may enter at least partially into the lumen 26 as the inner tubular body 20 is advanced into the region R. This may allow a portion of tissue from the region R to be retrieved, e.g., for a biopsy or other analysis.

Returning to FIG. 5C, once the distal end 22 of the inner tubular body 20 is desirably positioned within the region R, the agent is then delivered from the source 42 into the region R via the lumen 26 and distal end 22 of the inner tubular body 20. If the inner tubular body 20 includes one or more side ports 60, such as that shown in FIG. 4, the agent may exit from the side port(s) 60. As the agent is being delivered into the region R, some of the agent may seep or otherwise migrate into the tract 200. If the source 40 of vacuum is not already creating a vacuum within the lumen 18 of the outer tubular body 12, the source 40 may be activated to create a vacuum to aspirate the agent entering the tract 200 into the lumen 18 through the aspiration port(s) 28. Preferably, the source 40 of vacuum is activated before the agent delivery device 10 is inserted into the patient so that any fluid that enters the tract 200 is aspirated. Alternatively, the source 40 of vacuum may be activated at any time during the procedure, e.g., at periodic time intervals.

Turning to FIG. 5D, the inner tubular body 20 may be retracted proximally relative to the outer tubular body 12, e.g., to withdraw the distal end 22 of the inner member 20 into the outer tubular member 12. The agent delivery device 10 may then be withdrawn proximally from the tract 200 and the patient. If tissue is captured within the lumen 26 of the inner tubular body 20, it may be separated from the remaining tissue within the region R and removed from the patient as the device 10 is removed.

Prior to or while the delivery device is withdrawn, RF energy is then delivered by the generator 44 to the electrode 50 to coagulate, ablate, or otherwise treat the tissue surrounding the tract 200. In one method, only the tissue at region 202 adjacent the region R is treated, which should be sufficient to prevent migration of the agent from the region R and/or migration of tumor cells into the tract 200. Alternatively, energy may be delivered to additional tissue along the tract 200, i.e., to in short bursts such that spaced-apart regions are treated. In another alternative, energy may be delivered substantially continuously as the device 10 is withdrawn to substantially seal the tract 200 along its entire length. Thus, the tract 200 may be substantially sealed, thereby preventing or reducing the risk of track seeding from a tumor and/or contaminating tissue surrounding a target region to which an agent is delivered.

Although the agent delivery device has been described as having a rigid tubular body, the scope of the invention should not be so limited. In alternative embodiments, a flexible agent delivery device can be provided. For example, FIG. 6 shows an agent delivery device 300 in accordance with other embodiments of the invention. The agent delivery device 300 includes a tubular body 302 that has a proximal end 304, a distal end 306, and a lumen 308 extending between the proximal and the distal ends 304, 306. The tubular body 302 has a cross sectional dimension that is between 1.5 French to 7.0 French, and more specifically, between 2.5 French to 3.0 French. In other embodiments, the tubular body 302 can have other cross sectional dimensions. In the illustrated embodiments, the tubular body 302 is a catheter body that is made from an elastic material, such as PTFE, or other polymers, thereby allowing the tubular body 302 to flex or bend during use.

The agent delivery device 300 also includes an agent delivery port 328 that is in fluid communication with the lumen 308 of the tubular body 302. In the illustrated embodiments, the port 328 is located at a distal tip 329 of the tubular body 302. Alternatively, the port 328 is located proximal to the distal tip 329 and extends through a wall of the tubular body 302. Although one port 328 is shown, in alternative embodiments, the device 300 can have more than one port 328.

The agent delivery device 300 further includes an electrode 330 secured to the distal end 306 of the tubular body 302, and a braid 352 disposed within a wall 334 of the tubular body 302. The electrode 330 is electrically coupled to the braid 352. The electrode 330 can have a variety of configurations. For example, the electrode 330 can be a conductive marker band, a metallic deposit, or a coil. In the illustrated embodiment, the braid 352 is at least partially made from an electrically conductive material, and is used to provide strength for the tubular body 302, and to deliver current to the electrode 330. For example, the braid 352 can be made from one or more wires that are made from platinum-iridium, gold, silver, platinum, copper, or other conductive metals, polymers, or alloys. In the illustrated embodiments, the braid 352 has a braid density that ranges between approximately 80 and 150 pic count per unit length (number of intersections per unit length), but can also have other densities.

Although the braid 352 is shown in FIG. 6 (as well as the following Figures) as extending through a single tube, more complex braided designs can be used. In one embodiment, a braided layer of both insulative material and conductive material can be formed on the outside surface of a single tube layer or between two tube layers. For example, insulative strands can be interwoven with conductive strands using conventional braiding machines, and then the braided assembly thermally processed, so that the insulative strands melt and flow between the conductive strands. Further details describing the manufacture of braided tubes are disclosed in U.S. Pat. No. 6,635,047, which is expressly incorporated herein by reference.

Significantly, by using the braid 352 to provide strength for the tubular body 302, as well as providing a means for delivering RF energy to the electrode 330 (as opposed to using a separate RF wire), the tubular body 302 can have a relatively thinner wall, thereby providing a relatively small cross-sectional dimension for the agent delivery device 300. In other embodiments, a separate wire disposed within the wall 334 of the tubular body 302 can be used to deliver current to the electrode 330. In such cases, the braid 352 is optional, and the agent delivery device 300 may not include the braid 352. In still other embodiments, two or more conductive braids 352 can be used, in which case, the conductive braids 352 can be electrically isolated from each other by shifting their respective patterns by 180 degrees, so that braids 352 do not touch. In this case, the conductive braids 352 can be connected to two electrodes.

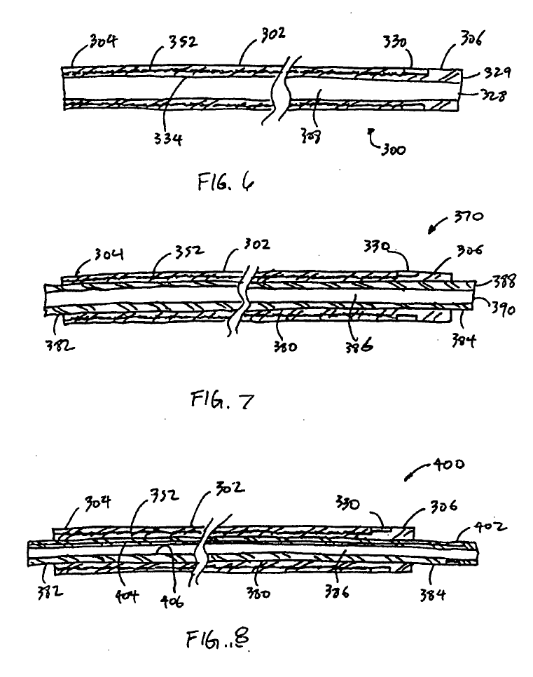

Although the agent delivery device 300 has been described as having a single tubular body, multiple tubular bodies can be provided in a similar manner described with respect to the agent delivery device 10. For example, FIG. 7 illustrates an agent delivery device 370 that includes the same outer tubular body 302 of FIG. 6, with the exception that the port 328 no longer acts as a drug delivery port, but rather serves as a distal port 328 from which an inner tubular body 380 deploys. The inner tubular body 380 is located coaxially within the lumen 308 of the outer tubular body 302, and is slidable relative to the tubular body 302. The inner tubular body 380 includes a proximal end 382, a distal end 384, and a lumen 386 extending between the proximal and the distal ends 382, 384. The inner tubular body 380 further includes a drug delivery port 390 located at a distal tip 388 of the inner tubular body 380. In the embodiment illustrated in FIG. 7, the tubular body 302 will typically have a cross-sectional dimension between 2.0 French and 7.0 French, and the inner tubular body 380 will have a cross-sectional dimension between 1.5 French and 6.5 French.

During use, the distal end 306 of the outer tubular body 302 may not be able to reach target tissue (e.g., because the diameter of the vessel adjacent the target site may be smaller than the cross sectional dimension of the outer tubular body 302). In such case, the distal end 384 of the inner tubular body 380 can be positioned distal to the distal end 306 of the outer tubular body 302 to reach target tissue that is distal to the distal end 306 of the outer tubular body 302. In the illustrated embodiments, the distal tip 388 is blunt. In other embodiments, the inner tubular body 380 can have a sharp distal tip 388, which can be used to pierce into target tissue. Also, in other embodiments, the port 390 is located proximal to the distal tip 388 and extends through a wall of the inner tubular body 380. Although one port 390 is shown, in alternative embodiments, the device 370 can have more than one port 390.

Although the agent delivery device 370 has been described as having a single electrode mounted to the outer tubular body, in other embodiments, electrodes can be mounted to the inner tubular body as well. For example, FIG. 8 illustrates an agent delivery device 400 that is the same as the device 370 of FIG. 7, except that it further includes a second electrode 402 secured to the distal end 384 of the inner tubular body 380, and a conductive braid 404 disposed within a wall 406 of the inner tubular body 380. The braid 404 is constructed in a similar manner as the previously described braid 352, and serves to both deliver current to the second electrode 402 from a generator (not shown) and strength, thereby minimizing the thickness of the wall 406.

The second electrode 402 can be selectively positioned relative to the first electrode 330 by positioning the inner tubular body 380 relative to the outer tubular body 302. In some embodiments, the device 400 further includes a marker (e.g., a radio opaque marker) secured to the outer tubular body 302 (or the inner tubular body 380) for allowing a physician to determine the position of the electrode 330 (and/or the electrode 402). In other embodiments, the device 400 can include a first marker secured to the outer tubular body 302, and a second marker secured to the inner tubular body 380. In the illustrated embodiment, the first electrode 330 is an active electrode, and the second electrode 402 is a return electrode, or vice versa, thereby allowing the agent delivery device 400 to deliver energy in a bipolar arrangement. Alternatively, both the first and the second electrodes 330, 402 are active electrodes, which delivery energy in a monopolar arrangement.

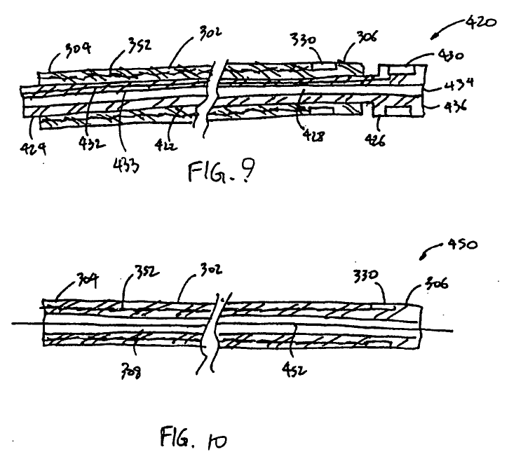

In the agent delivery device 400, the surface area of the second electrode is less than the surface area of the first electrode. Optionally, however, the surfaces areas of the first and second electrodes can be the same in order to provide a more consistent and efficient bipolar ablation. For example, FIG. 9 illustrates an agent delivery device 420 that includes the outer tubular body 302 of FIG. 6, and an inner tubular body 422 that has a proximal end 424, an enlarged distal end 426, and a lumen 428 extending between the proximal and distal ends 424, 426. The inner tubular body 422 further includes a drug delivery port 434 located at a distal tip 436 of the tubular body 422. In other embodiments, the port 434 can be located proximal to the distal tip 436, and/or the device 420 can include more than one port 434, as similarly discussed previously.

The agent delivery device 420 further includes a second electrode 430 secured to the enlarged distal end 426 of the inner tubular body 422, and a conductive braid 432 disposed within a wall 433 of the inner tubular body 380. The braid 404 is constructed in a similar manner as the previously described braid 352, and serves to both deliver current to the second electrode 430 from a generator (not shown), and strengthen and minimize the thickness of the wall 433. The second electrode 420 has an electrically conductive surface that has approximately the same surface area as that of the first electrode 330. In some embodiments, the device 420 can include a marker secured to the outer tubular body 302 (or the inner tubular body 422), or a marker secured to each of the outer and inner tubular bodies 302, 422, as similarly discussed previously.

In the above described agent delivery devices 400 and 420, a tubular body is used to carry the second electrode 402. However, the scope of the invention should not be so limited. In other embodiments, other structures can be used, to carry, or as, the second electrode 402. FIG. 10 illustrates an agent delivery device 450 in accordance with other embodiments of the invention. The device 450 includes the outer tubular body 302 of FIG. 6, and a wire 452 that is disposed within the lumen 308 of the tubular body 302. In such cases, the wire 452 is used as a second electrode, and is slidably disposed within the lumen 308 of the tubular body 302. In the illustrated embodiment, the first electrode 330 is an active electrode, and the wire 452 is a return electrode, or vice versa. Alternatively, the first electrode 330 and the wire 452 can be active electrodes. In some embodiments, portion(s) of the wire 452 can be covered by an insulative material, with the non-covered portion(s) of the wire 452 functioning as conductive region(s). Also, in other embodiments, instead of using the wire 452, the device 450 can include an elongate body, such as a solid shaft, to which a second electrode can be secured. In such cases, the elongate body is disposed within the lumen 308 of the tubular body 302, and is slidable relative to the tubular body 302 to adjust a distance between the first and the second electrodes.

Although some embodiments of the agent delivery device have been described has having a single electrode secured to a single structure, such as an outer tubular body or an inner tubular body, alternatively, any embodiments of the agent delivery device described herein can include a plurality of electrodes mounted to a single structure. In this case, the elements (e.g., wires) of the braid 352 can be covered with an insulating material such as Interrupted Layer Copolymer (ILC), thereby allowing one of the wires of the braid 352 to deliver energy to one electrode, and another of the wires of the braid 352 to delivery energy to, or return energy from, the other electrode(s).

If more than one electrode is provided, whether located on the inner tubular body or outer tubular body, the electrodes can be active electrodes, return electrodes, or combination thereof. For example, the electrodes on the outer tubular body can be active electrodes operative in association with one or more return electrodes that are either, placed exteriorly on a patient's skin, or secured to a structure (e.g., the inner tubular body) to ablate tissue. Alternatively, the electrodes on the outer tubular body can be return electrodes operative in association with one or more active electrodes that are secured to a structure (e.g., the inner tubular body). In further embodiments, one of the electrodes on the outer tubular body can be an active electrode, and another of the electrodes on the outer tubular body can be a return electrode. In some embodiments, one or more of the electrodes at the outer tubular body can be selectively switched (e.g., by a controller) to perform the function of either an active electrode or a return electrode.

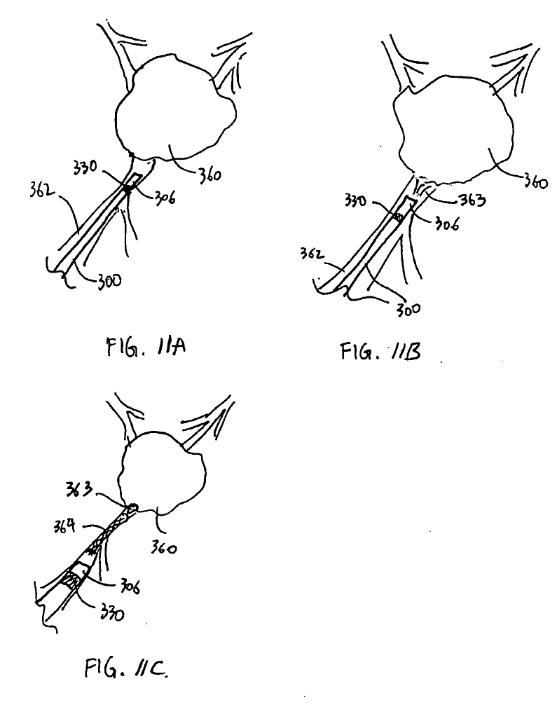

The above described flexible agent delivery device 300 can be used to treat tissue, such as a tumor, located at a remote target site in a patient. FIGS. 1A-11C illustrate a method of treating target tissue 360 using the agent delivery device 300. First, the distal end 306 of the tubular body 302 is inserted into a vessel 362, and is advanced distally until it reaches a target site (FIG. 11A). For example, a guidewire can be initially inserted into the patient, and is advanced distally through the blood vessel 362 to reach the target site. The tubular body 302 is then placed over the guidewire such that the guidewire is within the lumen 308 of the tubular body 302. In other methods, the agent delivery device 300 can further include a guidewire lumen disposed within the wall 334 of the tubular body 302. In such a case, the tubular body 302 is placed over the guidewire such that the guidewire is within the guidewire lumen. The tubular body 302 is then advanced distally, using the guidewire to steer the distal end 306 of the tubular body 302 to the target site. Alternatively, an introducer can be initially inserted into a patient to gain access to the target site. The distal end 306 of the tubular body 302 is then inserted into the introducer, and is advanced distally until the distal end 306 exits from a distal end of the introducer at the target site. In other methods, the agent delivery device 300 can itself be steered to the target site through blood vessels. For example, the agent delivery device 300 can further include one or more steering wires disposed within the wall 334 of the tubular body 302, with the distal end(s) of the steering wire(s) secured to the distal end 306 of the tubular body 302. The proximal end(s) of the steering wire(s) can be tensioned to bend the distal end 306, thereby steering the distal end 306 of the tubular body 302.

If any of the agent delivery devices 370, 400, or 420 is used, the method of introducing the outer tubular body 302 through the vessel 362 will be accomplished in the same manner. The distal end of the respective inner tubular body, however, will then be deployed out from the distal end of the outer tubular body until it reaches the target site. If the agent delivery device 450 is used, the electrode wire will be deployed out from the distal end of the outer tubular body until it reaches the target site. In any of the devices 370, 400, and 450, rather than using a separate introducer, the outer tubular body 302 can be used as an introducer for the respective inner tubular body.

Once the distal end 306 has been desirably placed at the target site, the proximal end 304 of the tubular body 302 is then coupled to a source of agent (not shown) such that the source of agent is in fluid communication with the delivery port 328 via the lumen 308, and coupled to a RF generator such that the RF generator is in electrical communication with the electrode 330 via the braid 352. Alternatively, if any of the agent delivery devices 370, 400, or 420 is used, the proximal end of the inner tubular body will be coupled to the source of agent (not shown), and the proximal end of the outer tubular body, and optionally, the inner tubular body, will be coupled to the RF generator. The agent 363 is then delivered from the source to the target site via the lumen 308 (FIG. 11B). For example, a drug, a medication, a toxic agent, or a treatment particle (e.g., a radiation seed or a micro toxic particle) can be delivered from the source to a tumor at the target site to treat the tumor.

After the agent has been delivered to the target site, the distal end 306 of the tubular body 302 is then retracted proximally to remove the tubular body 302 from the target site. Alternatively, if any of the agent delivery devices 370, 400, or 420 is used, the distal end of the inner tubular body will first be retracted into the distal end of the respective tubular body. If the agent delivery device 450 is used, the electrode wire will first be retracted into the outer tubular body. While the distal end 306 is retracted proximally, the electrode 330 can be energized by a generator, e.g., a radio frequency (RF) generator (not shown), to treat tissue at or adjacent the target site (FIG. 11C).

The electrode 330 delivers radio frequency electrical energy to coagulate, ablate, or otherwise treat the surrounding tissue to substantially seal or occlude at least a portion 364 of the vessel 362. In this method, the energy can be delivered in a monopolar arrangement, in which case, the electrode 330 functions as an active electrode that delivers the energy to the surrounding tissue, with a return electrode placed on the patient's skin to complete the current path. Alternatively, if either of the agent delivery devices 400 or 420 is used, the energy can be delivered in a bipolar arrangement between the electrode pair, or can be delivered in a monopolar arrangement from the electrode pair to a grounding pad.

In some methods, a conductive fluid can be delivered via the lumen 308, and exits from the port 328. The delivered conductive fluid can help transmit energy (e.g., ablation energy) from the electrode 330, and assists delivering of energy to the target tissue that otherwise cannot be reached directly by the electrode 330. In the illustrated method, only the portion of the vessel adjacent the target site is treated, which should be sufficient to prevent migration of the agent from the target site and/or migration of tumor cells into the vessel. In other methods, the portion of the vessel that is further away from the target site can also be treated. In either case, the vessel 362 may be substantially sealed, thereby preventing or reducing the risk of seeding from a tumor and/or contaminating tissue surrounding a target region to which the agent 363 is delivered. In some methods, the sealing of the vessel 362 also prevents blood from being supplied to the tumor, thereby preventing nutrients from being supplied to the tumor and further treating the tumor.

Although the agent delivery device 300 has been described as being used to access a treatment site via blood vessels, the scope of the invention should not be so limited. In alternative embodiments, the agent delivery device 300 can be used to access a treatment site via other paths or channels, such as through the medullary canal of a bone to reach bone tumors, such as those caused by Ewing's sarcoma.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. For example, any of the agent delivery devices described herein can include a suction lumen in fluid communication with a suction port, which can be used to vacuum or suck fluid away from a target site during use. In addition, an illustrated embodiment needs not have all the aspects or advantages of the invention shown. An aspect or an advantage described in conjunction with a particular embodiment of the present invention is not necessarily limited to that embodiment and can be practiced in any other embodiments of the present invention even if not so illustrated. Thus, the present invention is intended to cover alternatives, modifications, and equivalents that may fall within the spirit and scope of the present invention as defined by the claims.

1. An apparatus for delivering a medical substance to a target site within a body, comprising:
an outer tubular body having a distal end and a first lumen;
an inner tubular body having a distal end and a second lumen, wherein the inner tubular body is slidably disposed within the first lumen, such that the distal end of the inner tubular body can extend beyond the distal end of the outer tubular body;
a substance delivery port located on the distal end of the inner tubular body in fluid communication with the second lumen; and
a first electrode secured to the distal end of the outer tubular body.

2. The apparatus of claim 1, further comprising a second electrode secured to the distal end of the inner tubular body.

3. The apparatus of claim 1, further comprising a source of medical substance coupled to the second lumen.

4. The apparatus of claim 3, wherein the substance is a therapeutic agent.

5. The apparatus of claim 1, further comprising an aspiration port located on the outer tubular body.

6. The apparatus of claim 1, wherein the outer tubular body is a rigid shaft.

7. The apparatus of claim 1, wherein the outer tubular body is a flexible catheter body.

8. The apparatus of claim 1, wherein the inner tubular body is a needle.

9. A method for delivering a medical substance to a target site within a body, comprising:
advancing a tubular body through a channel to the target site;
delivering the substance via the tubular body to the target site; and
delivering ablation energy to seal the channel adjacent to the target site, whereby material is prevented from substantially migrating from the target site.

10. The method of claim 9, wherein the target site is a tumor.

11. The method of claim 9, further comprising aspirating at least some of the delivered substance from the channel or target site.

12. The method of claim 9, wherein the tubular body is a needle.

13. The method of claim 9, wherein the channel is a needle tract.

14. The method of claim 9, wherein the channel is a lumen of a blood vessel.

15. The method of claim 9, wherein the channel is a medullary canal of a bone.

16. The method of claim 9, wherein the material that is prevented from migrating from the target site is toxic.

17. The method of claim 9, wherein the material that is prevented from migrating from the target site is the medical substance.

18. The method of claim 9, wherein the ablation energy is electrical energy.

19. The method of claim 9, wherein the ablation energy is delivered from the tubular body.

20. A medical probe, comprising:
a probe shaft having a distal end, a tubular wall, and a lumen;
a first electrode secured to the distal end of the probe shaft;
a braided conductor extending through the wall of the probe shaft in electrical communication with the first electrode; and
a substance delivery port at the distal end of the probe shaft in fluid communication with the lumen.

21. The probe of claim 20, wherein the probe shaft comprises:
an outer tubular body formed by the tubular wall; and
an inner tubular body defining the lumen.

22. The probe of claim 21, wherein the electrode is secured to the outer tubular body, and the delivery port is located on the inner tubular body.

23. The probe of claim 22, further comprising a second electrode secured to the inner tubular body.

24. The probe of claim 20, wherein the probe shaft is flexible.
